(19) United States
(12) Patent Application Publication
(10) Pub. No.: US 2007/0036717 A1
(43) Pub. Date: Feb. 15, 2007

(54) Chemoradiotherapy with TS-1/camptothecins

(75) Inventors: Masahiko Watanabe, Kanagawa (JP); Takeo Sato, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: The Kitasato Gakuen Foundation

(21) Appl. No.: 11/498,098

(22) Filed: Aug. 3, 2006

(30) Foreign Application Priority Data
Aug. 11, 2005 (JP) 2005-233634

(51) Int. Cl. A61K 51/00; A61K 31/4745

(52) U.S. Cl. 424/1.11; 514/283

(57) ABSTRACT

An antitumor agent for chemoradiotherapy of rectal cancer comprising a combination of TS-1 (a combination drug of tegafur, gimeracil, and oteracil potassium at a 1:0.4:1 molar ratio) and CPT-11 (irinotecan hydrochloride). The antitumor agent of the invention can achieve marked tumor volume reduction without causing major side effects, especially by coadministering it with preoperative radiation therapy. The volume of even large tumors that are refractory to surgical resection can be reduced by coadministering the antitumor agent of the invention with preoperative radiation therapy, making the subsequent surgical resection of the tumor easier.

### TECHNICAL FIELD

The present invention relates to an antitumor agent useful for chemoradiotherapy Comprising a combination of tegafur, gimeracil, oteracil potassium and camptothecin or a camptothecin derivative, a combination therapy of said antitumor agent and radiation therapy, and an antitumor preparation and a kit for treating cancer, comprising said antitumor agent.

### BACKGROUND ART

In recent years, the incidence of colorectal cancer in Japan has increased year by year and now rivals rates in the West. There were 49,739 deaths from colorectal cancer in 1997 (accounting for 12.1% of all deaths from malignant neoplasms), and 79,404 people (128.6 in 100,000) develop colorectal cancer annually. One estimate of cancer rates in 2015 predicts that the number of people with colorectal cancer (colon cancer+rectal cancer) will reach almost 170,000 and that colorectal cancer will replace stomach and lung cancer as the No. 1 cancer (Cancer Statistics in Japan, January 2001).

A standard therapy for rectal cancer has yet to be established. Numerous clinical tests of radiation therapy as an adjuvant to surgery have been conducted in western countries. Among them, GITSG 7175, postoperative chemoradiotherapy improved relapse-free survival rate compared to resection alone (New England Journal of Medicine, Vol. 312, No. 23, 1465-1472, 1985). In light of these results, the United States National Institutes of Health currently recommends usual resection plus postoperative chemoradiotherapy as standard therapy for P-Stage II and III rectal cancer (Journal of the American Medical Association, Vol. 264, No. 11, 1444-1450, 1990). It was later reported in NSABP R02 that although postoperative chemoradiotherapy increases local suppression rates, it does not contribute to relapse-free survival or overall survival (Journal of the National Cancer Institute, Vol. 92, No. 5, 388-396, 2000). In contrast, reports of cases of pathological complete response (pCR) with preoperative chemoradiotherapy contributing to increased relapse-free survival rates have begun to draw attention in recent years. In addition to providing an antitumor effect, it is important that therapy for advanced cancer ensure a good postoperative quality of life (QOL), and reducing tumor volume by preoperative chemoradiotherapy increases the potential for performing radical surgery that preserves anal function.

Various effective novel antitumor agents have been introduced for preoperative chemoradiotherapy, and various strategies intended to increase survival rates in rectal cancer have been implemented. Known examples of such treatment strategies include radiation therapy with adjuvant 5-fluorouracil/irinotecan hydrochloride (5-FU/CPT-11) (Int. J. Radiat. Oncol. Biol. Phys., Vol. 55, No. 1, 132-137, 2003; Proc. Am. Soc. Clin. Oncol. #1052, 2003; Proc. Am. Soc. Clin. Oncol. #3612, 2004; British Journal of Cancer, Vol. 92, No. 7, 1215-1220, 2005), radiation therapy with adjuvant capecitabine/irinotecan hydrochloride (capecitabine/CPT-11) (Journal of Clin. Oncol., Vol. 23, No. 7, 1350-1357, 2005), and irinotecan hydrochloride (CPT-11) and radiation therapy (International Patent Publication No. 2000/61187).

However, the conventional preoperative chemoradiotherapies do not afford sufficient tumor volume reduction and entail strong side effects. Therefore, a novel preoperative chemoradiotherapy that is more highly effective, has fewer side effects, and maintains the patient's QOL is desired.

### DISCLOSURE OF THE INVENTION

Thus, the object of the present invention is to provide a novel chemoradiotherapy that affords a high level of tumor volume reduction and causes few side effects. In particular, the object of the invention is to provide an antitumor agent for use in preoperative chemoradiotherapy, a therapy using said antitumor agent, and a pharmaceutical formulation and a kit comprising said antitumor agent.

As a result of extensive research on novel chemoradiotherapies aimed at enhancing tumor volume reduction and radiation sensitivity, the inventors discovered that tumor volume reduction is markedly enhanced by the coadministration of a tegafur-gimeracil-oteracil potassium combination drug (molar ratio of tegafur:gimeracil:oteracil potassium=1:0.4:1, manufactured by Taiho Pharmaceutical Co., Ltd.; referred to hereinbelow as TS-1), the camptothecin derivative irinotecan hydrochloride (manufactured by Yakult Honsha Co., Ltd.; referred to hereinbelow as CPT-11), and radiation therapy.

It is expected that the coadministration of the antitumor agent of the invention and radiation therapy will markedly reduce tumor volume, reduce side effects associated with therapy, shorten intravenous infusion time, and reduce the number of clinic visits for the patient. In addition, the inventors confirmed that the tumor volume reduction of this novel chemoradiotherapy is equal or superior to that of conventional preoperative chemoradiotherapies and that the side effects associated with the novel chemoradiotherapy are much milder than the side effects associated with conventional preoperative chemoradiotherapies, thus leading to the present invention.

Thus, the present invention concerns the following chemoradiotherapy for rectal cancer, antitumor agent for rectal cancer, and kit for chemoradiotherapy of rectal cancer.
1. Chemoradiotherapy for rectal cancer, comprising radiation irradiation in combination with administration of tegafur in a therapeutically effective amount, gimeracil in an amount effective for potentiating an antitumor effect, oteracil potassium in an amount effective for inhibiting side effects, and camptothecin or a camptothecin derivative in a therapeutically effective amount.
2. The chemoradiotherapy according to 1 above, wherein the camptothecin derivative is irinotecan hydrochloride.
3. The chemoradiotherapy according to 1 above, wherein the molar ratio of tegafur, gimeracil, and oteracil potassium is 1:0.1˜5:0.1˜5.
4. The chemoradiotherapy according to 3 above, wherein the molar ratio of tegafur, gimeracil, and oteracil potassium is 1:0.4:1.
5. The chemoradiotherapy according to 4 above, wherein a combination drug of tegafur, gimeracil and oteracil potassium with a molar ratio of 1:0.4:1 is administered in an amount of about 10 to about 200 mg/m²/day, based on the tegafur; irinotecan hydrochloride is administered in an amount of about 10 to about 200 mg/m²/day; and the radiation dose is in a range of 40 to 60 Gy per course.
6. The chemoradiotherapy according to 5 above, wherein the combination drug containing tegafur, gimeracil and oteracil potassium at a molar ratio of 1:0.4:1 is administered in an amount of about 80 mg/m²/day, based on the tegafur, on Days 1 to 5, 8 to 12, 22 to 26, 29 to 33; the irinotecan hydrochloride is administered in an amount of about 80 mg/m²/day on Days 1, 8, 22 and 29; and 1.8 Gy/day of radiation is administered on Days 1 to 5, 8 to 12, 15 to 19, 22 to 26 and 29 to 33.
7. The chemoradiotherapy according to any one of 1 to 6 above, wherein the disease stage of the patient to undergo treatment is T3 or T4 in terms of depth of wall invasion and falls within a range of N0 to N3 in terms of lymph node metastasis according to the rectal cancer staging system.
8. The chemoradiotherapy according to 7 above, which is performed prior to surgical resection of the tumor.
9. An antitumor agent for use in chemoradiotherapy of rectal cancer, comprising a combination of tegafur in a therapeutically effective amount, gimeracil in an amount effective for potentiating an antitumor effect, oteracil potassium in an amount effective for inhibiting side effects, and camptothecin or a camptothecin derivative in a therapeutically effective amount.
10. The antitumor agent according to 9 above, wherein the camptothecin derivative is irinotecan hydrochloride.
11. The antitumor agent according to 9 or 10 above, wherein the molar ratio of tegafur, gimeracil and oteracil potassium is 1:0.1˜5:0.1˜5.
12. The antitumor agent according toll above, wherein the molar ratio of tegafur, gimeracil and oteracil potassium is 1:0.4:1.
13. The antitumor agent according to any one of 9 to 12 above, which is used for preoperative chemoradiotherapy of rectal cancer.
14. An antitumor agent for use in chemoradiotherapy of rectal cancer, which is a pharmaceutical formulation consisting of two or more preparations containing tegafur, gimeracil, oteracil potassium and camptothecin or a camptothecin derivative, which preparation each comprises one or any combination of said active ingredients, or which is a pharmaceutical formulation consisting of a single preparation containing all of the active ingredients.
15. The antitumor agent according to 14 above, wherein the camptothecin derivative is irinotecan hydrochloride.
16. The antitumor agent according to 14 or 15 above, wherein the molar ratio of tegafur, gimeracil and oteracil potassium is 1:0.1˜5:0.1˜5.
17. The antitumor agent according to 16 above, wherein the molar ratio of tegafur, gimeracil and oteracil potassium is 1:0.4:1.
18. The antitumor agent according to any one of 14 to 17 above, which comprises a combination drug containing tegafur, gimeracil and oteracil potassium as the active ingredients and a preparation containing irinotecan hydrochloride as the active ingredient.
19. The antitumor agent according to any one of 14 to 18 above, which is used for preoperative chemoradiotherapy for rectal cancer.
20. A kit for use in chemoradiotherapy of rectal cancer, which comprises a combination of (a) a combination drug containing tegafur in a therapeutically effective amount, gimeracil in an amount effective for potentiating an antitumor effect, oteracil potassium in an amount effective for inhibiting side effects, and (b) a preparation of camptothecin or a camptothecin derivative in a therapeutically effective amount.
21. The kit according to 20 above, wherein the camptothecin derivative is irinotecan hydrochloride.
22. The kit according to 20 or 21 above, wherein the molar ratio of tegafur, gimeracil, and oteracil potassium is 1:0.1˜5:0.1˜5.
23. The kit according to 22 above, wherein the molar ratio of tegafur, gimeracil, and oteracil potassium is 1:0.4:1.
24. The kit according to any one of 20 to 23 above, which is used for preoperative chemoradiotherapy for rectal cancer.

The active antitumor ingredient tegafur (generic name) is a known compound (chemical name: 5-fluoro-1-(2-tetrahydrofuryl)-2,4-(1H,3H)-pyrimidinedione; abbreviated as FT hereinbelow). Tegafur is activated in vivo where it releases 5-FU, the substance with antitumor activity. Tegafur can be manufactured by methods known in the art, for example, as described in Japanese Examined Patent Publication (Tokko) No. 1974-10510.

Gimeracil (generic name) is also a known compound (chemical name: 2,4-dihydroxy-5-chloropyridine; abbreviated as “CDHP” hereinbelow). Gimeracil itself has no antitumor activity, but suppresses the metabolism and deactivation of 5-FU in the body and thereby can potentiate antitumor effect.

Oteracil potassium (generic name) is also a known compound (chemical name: monopotassium. 1,2,3,4-tetrahydro-2,4-dioxo-1,3,5-triazine-6-carboxylate; abbreviated as “OXO” hereinbelow.) Oteracil potassium itself possesses no antitumor activity, but is chiefly distributed throughout the gastrointestinal tract where it controls adverse gastrointestinal effects by suppressing 5-FU activity.

The active antitumor ingredient camptothecin (generic name) is also a known compound (chemical name: (45)-4-hydroxy-4-ethyl-1H-pyrano[3′,4′:6,7]indolizino[1,2-b]quinoline-3,14(4H,12H)-dione). Camptothecin kills cancer cells by inhibiting DNA topoisomerase I, thereby suppressing DNA synthesis and transcription. Camptothecin can be manufactured by methods known in the art, for example, as described in Japanese Examined Patent Publication (Tokko) No. 1982-27113.

Examples of the camptothecin derivative, an active antitumor ingredient, include natural-occurring derivatives such as 10-hydroxycamptothecin, 11-hydroxycamptothecin, 9-methoxycamptothecin, 10-methoxycamptothecin and 11-methoxycamptothecin as well as camptothecin derivatives obtained by semisynthetic processes by modifying raw materials such as said natural camptothecin (e.g., irinotecan, topotecan, rubitecan (RFS-2000), exatecan (DX-8591f), CKD-602, DRF-1042, lurtotecan (NX-211), karenitecin (BNP-1350), silatecan (DB-67), diflomotecan (BN-80915), gimatecan (ST-1481), BAY-38-3441, MAG-CPT (PNU-166148), CT-2106, prothecan and DE-310), or their pharmaceutically acceptable salts. Among these, irinotecan or its pharmaceutically acceptable salts are preferred. More preferred is irinotecan hydrochloride ((4S)-4a, 11-diethyl-4β-hydroxy-9-[[4-(piperidin-1-yl)piperidin-1-yl]carbonyloxy]1H-pyrano[3′,4′:6,7]indolizino[1,2-b]quinoline-3,14-(4H,12H)dione hydrochloride salt; abbreviated as “CPT-11” hereinbelow).

The active antitumor agent CPT-11 is a known compound that suppresses DNA synthesis and transcription by inhibiting DNA topoisomerase I, thereby causing cancer cells to die. CPT-11 can be manufactured by methods known in the art such as that described in Japanese Examined Patent Publication (Tokko) 1991-4077 (1991).

In the present invention, tegafur, gimeracil, oteracil potassium and CPT-11 can be combined into antitumor agent. This antitumor agent is pharmaceutical formulation consisting of two or more preparations containing tegafur, gimeracil, oteracil potassium and camptothecin or a camptothecin derivative, which preparation each comprises one or any combination of said active ingredients, or which is a pharmaceutical formulation consisting of a single preparation containing all of the active ingredients. Thus, the antitumor agent of the invention may be a single-preparation combination drug containing all four of the above ingredients, or it may be a multiple-preparation combination drug comprising a combination drug containing 1 to 3 of the ingredients and other combination drugs containing the other ingredients. Especially preferably, the formulation consists of 2 drugs, which are TS-1 which contains tegafur, gimeracil and oteracil potassium as the active ingredient at a molar ratio of 1:0.4:1 and another combination drug containing CPT-11 as the active ingredient.

The ratio of the active ingredients, tegafur, gimeracil, and oteracil potassium contained in the combination drug of the invention may be the same range as, for example, that of the known combination drug described in Japanese Patent 2,614,164. This ratio is usually about 0.1 to about 5 moles, preferably about 0.2 to about 1.5 moles, of gimeracil and about 0.1 to about 5 moles, preferably about 0.2 to about 2 moles, of oteracil potassium, based on 1 mole of tegafur. It is particularly preferred that the combination drug of three ingredients have a molar ratio of tegafur:gimeracil:oteracil potassium equaling 1:0.4:1.

The combination drug of the invention having tegafur, gimeracil and oteracil potassium as the three active ingredients may be either a pharmaceutical formulation comprising plural preparations, each containing one or any combination of the respective ingredients or a pharmaceutical formulation comprising a single preparation containing all of the active ingredients. In either case, pharmaceutical compositions can be prepared by conventional methods, using suitable pharmaceutical vehicles. Here, examples of vehicles used include those commonly and conventionally used in drug preparations, for example, excipients, binders, disintegrators, lubricants, colorants, flavoring agents and surfactants. When a combination drug comprising two or more of the above combination preparations and having tegafur, gimeracil and oteracil potassium as the three active ingredients is used, all of the ingredients may be administered simultaneously or the remaining ingredients may be administered at any time before or after the first ingredient. Preferably, the ingredients should be administered simultaneously or the others should be administered within 4 hours before or after administering the first ingredient, more preferably within 2 hours of administering the first ingredient.

There are no particular restrictions on the form of the dosage unit for the combination drug of the invention having tegafur, gimeracil and oteracil potassium as the three active ingredients when the combination drug is used for treatment of malignant tumors in mammals, including humans. A form that is suitable for the treatment objective can be selected. Specific examples include non-oral forms such as injections, suppositories, ophthalmic solutions, ointments, and aerosols and oral forms such as tablets, coated tablets, powders, granules, capsules, liquids, pills, suspensions, and emulsions, with oral forms being preferred. The above dosage forms are manufactured by methods conventionally known in this field.

When the preparation of the invention having CPT-11 as the active ingredient is administered separately or simultaneously with the above-described combination drug having tegafur, gimeracil, and oteracil potassium as the three active ingredients, the dose of CPT-11 should be in the range of, for example, about 0.01 to about 0.8 moles based on 1 mole of tegafur, with about 0.1 to about 0.4 moles being preferred, and about 0.2 to about 0.3 moles being more preferred.

The preparation with CPT-11 as the active ingredient of the invention is formulated as any of various types of independent dosage unit. In this case, a pharmaceutical composition can be made by conventional methods, using suitable pharmaceutical vehicles. Examples of the vehicles used here include those commonly and conventionally used in drug preparations, for example, excipients, binders, disintegrators, lubricants, colorants, flavoring agents and surfactants. The preparation having CPT-11 as the active ingredient formulated into any of various dosage unit forms can be administered separately or simultaneously with the combination drug having tegafur, gimeracil, and oteracil potassium as the three active ingredients which is also formulated into any of various dosage unit forms. Thus, the preparation having CPT-11 as the active ingredient can be administered at any time, before, after, or simultaneously with administering the combination drug having tegafur, gimeracil, and oteracil potassium as the three active ingredients. Preferably, it should be administered simultaneously or within 4 hours, more preferably within 2 hours, before or after administering the combination drug having tegafur, gimeracil, and oteracil potassium as the three active ingredients.

There are no particular restrictions on the form of the dosage unit for the inventive preparation having CPT-11 as the active ingredient when used for treatment of malignant tumors in mammals, including humans A form that is suitable for the treatment objective can be selected. Specific examples include non-oral forms such as injections, suppositories, ophthalmic solutions, ointments, and aerosols and oral forms such as tablets, coated tablets, powders, granules, capsules, liquids, pills, suspensions, and emulsions, with injections being the preferred form. The above dosage forms are manufactured by methods conventionally known in this field.

There are no particular restrictions on the blending ratio of the antitumor agent of the invention, regardless of whether it is a single preparation or plural preparations. However, it should usually be about 0.1-5 moles, preferably about 0.2-1.5 moles, of gimeracil, about 0.1-5 moles, preferably about 0.2-2 moles, of oteracil potassium, and about 0.01-0.8 mole, preferably about 0.1-0.4 mole, more preferably 0.2-0.3 mole of CPT-11 per mole of tegafur. In particular, the preferred molar ratio of each ingredient is tegafur:gimeracil:oteracil potassium:CPT-11 equaling about 1:0.4:1:0.01-0.8, with about 1:0.4:1:0.1-0.4 moles being more preferred, and about 1:0.4:1:0.2-0.3 moles being even more preferred. When the above-described combination drug having tegafur, gimeracil, and oteracil potassium as the three active ingredients and the preparation having CPT-11 as the active ingredient are two separate preparations, which is the preferred mode, it is preferred that the molar ratio of tegafur:gimeracil:oteracil potassium in the combination drug be 1:0.4:1 and that the CPT-11 preparation contain about 0.01-1 mole, preferably about 0.1-0.4 mole, more preferably about 0.2-0.3 mole of CPT-11 per mole of tegafur.

Pharmaceutical compositions can be made from these active ingredients by conventional methods, using suitable pharmaceutical vehicles. Examples of the vehicles used here include those commonly and conventionally used in drug preparations, for example, excipients, binders, disintegrators, lubricants, colorants, flavoring agents and surfactants.

When a plural-preparation type antitumor agent comprising at least two preparations, as described hereinabove, is used, all of the ingredients may be administered simultaneously, alternatively before or after some of the ingredients are administered, the other ingredients may be administered at any time. Preferably, all the ingredients are administered simultaneously, alternatively, before or after administering the first ingredients), the others are administered within 4 hours, more preferably within 2 hours.

There are no particular restrictions on the form of the dosage unit when the antitumor agent of the invention is used for treatment of malignant tumors in mammals, including humans. A suitable form can be selected according to the treatment objective. Specific examples of the dosage form include non-oral forms such as injections, suppositories, ophthalmic solutions, ointments and aerosols and oral forms such as tablets, coated tablets, powders, granules, capsules, liquids, pills, suspensions, and emulsions. For administration of preparations of a combination of tegafur, gimeracil and oteracil potassium, oral form is preferred and for administration of a preparation containing CPT-11 as the active ingredient, injection is preferred. The above dosage forms are manufactured by methods conventionally known in this field.

Examples of vehicles that may be used when preparing solid oral preparation such as tablets, powders and granules include the following: excipients such as lactose, sucrose, sodium chloride, glucose, urea, starch, calcium carbonate, kaolin, crystalline cellulose, silicic acid, methyl cellulose, glycerol, sodium alginate and gum arabic; binders such as simple syrup, glucose solutions, starch solution, gelatin solution, poly(vinyl alcohol), poly(vinyl ether), poly(vinyl pyrrolidone), carboxymethylcellulose, shellac, methylcellulose, ethylcellulose, water, ethanol and potassium phosphate; disintegrators such as dry starch, sodium alginate, agar powder, laminaran powder, sodium hydrogen carbonate, calcium carbonate, polyoxyethylene sorbitan fatty acid esters, sodium lauryl sulfate, stearic acid monoglyceride, starch and lactose; disintegration inhibitors such as sucrose, stearic acid, cacao butter and hydrogenated oil; absorption promoters such as quaternary ammonium bases and sodium lauryl sulfate; humectants such as glycerol and starch; adsorbents such as starch, lactose, kaolin, bentonite, and colloidal silicic acid; and lubricants such as purified talc, stearic acid salts, boric acid powder, and polyethylene glycol. In addition, tablets with conventional coatings may be used as desired, for example, sugar-coated tablets, gelatin-coated tablets, enteric-coated tablets, film-coated tablets, double- and multi-layer tablets.

The following are examples of vehicles that may be used in the preparation of pills: excipients such as glucose, lactose, starch, cacao butter, hydrogenated vegetable oil, kaolin and talc; binders such as gum arabic powder, tragacanth powder, and gelatin; and disintegrators such as laminaran and agar.

Capsules are prepared by mixing the active ingredients with various vehicles, examples of which are given hereinabove, and filling them in hard gelatin capsules, soft capsules, and the like.

Examples of vehicles that may be used when preparing suppositories include polyethylene glycol, cacao butter, lanolin, higher alcohols, higher alcohol esters, gelatin, semisynthetic glycerides and Witepsol (registered trademark, Dynamit Nobel AG).

Examples of vehicles that may be used when preparing injections include diluents such as water, ethyl alcohol, macrogol, propylene glycol, ethoxylated isostearyl alcohol, polyoxylated isostearyl alcohol, and polyoxyethylene sorbitan fatty acid ester; pH adjustors such as sodium citrate, sodium acetate, and sodium phosphate; buffering agents such as dipotassium hydrogen phosphate, trisodium phosphate, sodium hydrogen phosphate and sodium citrate; stabilizers such as sodium pyrosulfite, EDTA, thioglycolic acid and thiolactic acid; and binders for lyophilization, for example, sugars such as sorbitol, mannose, mannitol, inositol, maltose, sucrose and lactose. An adequate amount of sodium chloride, glucose or glycerol may also be added to the pharmaceutical preparation to prepare an isotonic solution and also, conventional solubilization aids, soothing agents, local anesthetics and the like may also added. Subcutaneous, intramuscular and intravenous injections can be manufactured by conventional methods, with the addition of these vehicles.

Liquid preparations may be aqueous or oil-based suspensions, solutions, syrups or elixirs prepared by conventional methods, using conventional additives.

When preparing ointments, for example, in the form of pastes, creams and gels, diluents such as white petrolatum, paraffin, glycerol, cellulose derivatives, polyethylene glycol, silicone, or bentonite may be used.

There are no particular restrictions on the amounts of tegafur, gimeracil, oteracil potassium and CPT-11, the active ingredients in the antitumor agent of the invention, blended into the antitumor agent. Suitable amounts should be selected in view of considerations such as dosage form, route of administration, and treatment plan, but usually it is preferable that the amount of each of the ingredients be about 1 to 70 percent by mass in the agent.

In the present invention, the above-described preparation containing CPT-11 and combination drug of tegafur, gimeracil, and oteracil potassium can be made into a kit comprising a combination of

(a) a combination drug containing tegafur in a therapeutically effective amount, gimeracil in an amount effective for potentiating an antitumor effect, and oteracil potassium in an amount effective for inhibiting side effects and

(b) a preparation containing CPT-11 in a therapeutically effective amount.

The respective compositions constituting this kit may be any pharmaceutical formulations known in the art, and each composition is generally kept in the type of container conventionally used for that type of pharmaceutical formulation.

In addition, this kit can be, for example, a kit for therapeutic use for cancer in mammals that contains at least four ingredients comprising a combination drug of

(i) tegafur in a therapeutically effective amount,

(ii) gimeracil in an amount effective for potentiating antitumor effect, and

(iii) oteracil potassium in an amount effective for inhibiting side effects,

as well as

(iv) CPT-11 in a therapeutically effective amount. This kit can also include at least two containers for these ingredients wherein the tegafur and CPT-11 are kept in different containers. It is preferred that the above-mentioned ingredients (i) to (iv) be in pharmaceutical formulations wherein they are each combined with pharmaceutically acceptable vehicles. In the above-mentioned kit, ingredients (i) and (iv) should be kept in separate containers, while ingredients (ii) and (iii) may each be kept in separate containers from the two above-mentioned ingredients, and either may be mixed with ingredient (i) or (iv) and kept in the same container with it. In the kit, it is preferred to keep the preparation containing ingredients (i)-(iii) in one container and the preparation containing ingredient (iv) in another container.

There are no particular restrictions on the method of administration of the antitumor agent of the invention; an appropriate method may be determined on the basis of the pharmaceutical formulation, age, sex and other conditions regarding the patient, and severity of the patient's symptoms. Examples include enteral administration, oral administration, rectal administration, intraoral administration, intraarterial and intravenous administration, and percutaneous administration. For example, tablets, pills, liquids, suspensions, emulsions, granules, capsules and the like are administered orally; injections are administered intraarterially or intravenously; suppositories are administered intrarectally; and ointments are applied to the skin, intraoral mucous membrane or the like. With the antitumor agent of the invention, it is also possible, for example, to administer the combination drug having tegafur, gimeracil, and oteracil potassium as the three active ingredients orally and the preparation having CPT-11 as the active ingredient as an intravenous injection.

The dose of each active ingredient in the present invention can be selected as appropriate for such as administration, age and sex of the patient, disease stage, and other conditions. The administration of antitumor agent of the invention can also be divided into 1 to 4 different doses a day.

As for oral administration, it is preferable that the daily dosage of tegafur be about 10 to about 200 mg/m²/day, preferably about 60 to about 120 mg/m²/day, more preferably about 80 mg/m²/day, that the dosage of gimeracil be about 2 to 58 mg/m²/day, preferably about 17 to about 35 mg/m²/day, more preferably about 23 mg/m²/day, that the dosage of oteracil potassium be about 9 to about 196 mg/m²/day, preferably about 58 to about 118 mg/m²/day, more preferably about 78 mg/m²/day, and that the dosage of CPT-11 be about 10 to about 200 mg/m²/day, preferably about 30 to about 100 mg/m²/day, more preferably about 80 mg/m²/day. These dosages are daily amounts and each dosage amount can be divided into 2 to 4 doses, preferably 2 doses per day.

As for injections, the usual daily adult dosage amount of tegatur (about 10 to about 200 mg/m²) and the usual adult daily dosage of CPT-11 (about 10 to about 200 mg/m², preferably about 30 to about 100 mg/m²/day, more preferably about 80 mg/m²/day) can be diluted with isotonic saline solution or glucose injection and slowly administered over at least 5 minutes, preferably 60 minutes, more preferably 90 minutes.

As for suppositories, the usual adult daily dosage of about 10 to about 200 mg/m², based on the tegafur, and about 10 to about 200 mg/m² of CPT-11 can be administered by insertion in the rectum once a day or twice a day at an interval of 6 to 12 hours.

The type of tumor that can be treated with the antitumor agent of the invention is rectal cancer. An even greater effect can be expected when the disease stage for rectal cancer is a depth of wall invasion of T3 or T4 and lymph node metastases of N0 to N3 according to the Japanese Classification of Colorectal Carcinoma, Sixth Edition, Japanese Society for Cancer of the Colon and Rectum, Kanehara Co., Ltd. (Tokyo), 2000 version.

By coadministering the antitumor agent of the invention and radiation therapy, it is possible to obtain marked tumor volume reduction, compared to conventional chemoradiotherapy, especially conventional preoperative chemoradiotherapy, without the accompanying side effects. Radiation therapy generally used in the applicable technical field can be used in combination with the antitumor agent of the invention, following protocols known to those conversant in the art. For example, cesium, iridium, iodine and cobalt irradiation are included in the above irradiation therapies. Local irradiation of tissue at the tumor site is preferable. A course of radiation therapy is conducted over 20 to 30 sessions (about 4 to 6 weeks), preferably 25 sessions (5 weeks). The radiation dose per course is preferably 40 to 60 Gy, more preferably 45 Gy.

The antitumor agent of the invention and radiation therapy may be coadministered simultaneously or at different times. It is preferred to administer radiation therapy within 4 hours, more preferably within 2 hours, before or after administering the antitumor agent of the invention.

Combination therapy with the antitumor agent of the invention and radiation therapy affords marked tumor volume reduction. Eradication of tumors can be expected with the chemoradiotherapy of the invention alone without surgical resection. An even greater therapeutic effect can be expected by combining the chemoradiotherapy of the invention and surgical resection of the tumor. Here, the chemoradiotherapy of the invention maybe preoperative chemoradiotherapy performed before surgical resection of the tumor, postoperative chemoradiotherapy performed after surgical resection of the tumor, and chemoradiotherapy performed both before and after surgical resection of the tumor. A particular advantage of the chemoradiotherapy of the invention is that surgical resection of the tumor will be easier after performing the chemoradiotherapy because the therapy affords marked tumor volume reduction. Furthermore, it is preferable to perform the chemoradiotherapy of the invention preoperatively because the potential for performing radical surgery that preserves anal function is increased, which is desirable in terms of the patient's QOL. More preferred is performing surgery after at least 4 to 6 weeks have elapsed since the chemoradiotherapy of the invention. There are no particular restrictions on the surgery here, and any surgical technique known to those skilled in the art can be used. For example, abdominoperineal resection of the rectum and super low anterior resection of the rectum can be performed.

The antitumor agent of the invention can achieve marked tumor volume reduction without causing major side effects, especially by coadministering it with preoperative radiation therapy. The volume of even large tumors that are refractory to surgical resection can be reduced by coadministering the antitumor agent of the invention with preoperative radiation therapy, making the subsequent surgical resection of the tumor easier.

### BEST MODE FOR CARRYING OUT THE INVENTION

The following examples will be used to describe the present invention in further detail; however, the invention is not limited by these examples.

### EXAMPLES

### Example 1

(1) Purpose

A Phase I/II clinical study of preoperative chemoradiotherapy with TS-1/CPT-11 was conducted to estimate the maximum tolerated dose (MTD) and recommended dose (RD) of CPT-11. The subjects had cancer of the rectum (excluding the rectosigmoid (Rs)) with a preoperative diagnosis of T3′ (tumors showing invasion through the muscularis propria into the subserosa, or into the nonperitonealized pericolic or perirectal tissues) or T4′ (tumors perforating the visceral peritoneum and/or directly invading other organs or structures), and N0 to N3′. Response rates and safety (percentage of adverse reactions and percentage of complications) were also evaluated with RD administration. (2) Subject selection

A total of 18 subjects, 15 male patients of age 46 to 80 years and 3 female patients of age 55 to 57 years, were selected on the basis of the above criteria.

(3) Treatment Plan

One course of chemoradiotherapy was 35 days.

The standard radiation regimen is 5 continuous weeks with a 1.8 Gy/day dose of irradiation five days a week and two days off a week (45 Gy total). Single-field irradiation was administered, and only the tumor site was irradiated (lymph nodes were not irradiated).

From two days preceding TS-1/CPT-11 administration day to the administration day and on Days 6 to 8, Days 20 to 22, and Days 27 to 29, the subjects were observed for diarrhea, and a suitable amount of the crude drug preparation hange-shashinto was administered orally. Maglax or magnesium oxide was given to control bowel movements in patients developing constipation as a result of the hange-shashinto. Suitable amounts of the following were administered: Decadron (an antinflammatory) intravenously and Kytril (an antiemetic) intravenously on Days 1, 8, 22, and 29, Kytril orally on Days 2 to 4, 9 to 11, 23 to 24 and 30 to 32, and Primperan (an antiemetic) orally from the first day (Day 1) to the last day (Day 35) of TS-1 and CPT-11 administration.

TS-1 was administered orally after breakfast and dinner for five days with two days off, on Days 1 to 5, 8 to 12, 22 to 26 and 29 to 33, according to the dosage schedule based on body surface area in Table 1 hereinbelow.

TABLE 1

Dosage Regimens
(80 mg/m²/day)

| Body Surface Area | Basic Dose | Basic Daily Dose |
|---|---|---|
| <1.25 m² | 40 mg/dose | 80 mg/day |
| ≧1.25 m² to <1.5 m² | 50 mg/dose | 100 mg/day |
| ≧1.5 m² | 60 mg/dose | 120 mg/day |

A dose of 30 to 100 mg/m² of CPT-11, mixed with at least 500 mL of isotonic saline solution, 5 percent glucose solution, or electrolyte-maintenance solution was administered by intravenous infusion over a period of at least 90 minutes on Days 1, 8, 22 and 29, according to the ascending dose titration schedule hereinbelow. CPT-11 should be administered as soon as possible after mixing with the parenteral solution.

Surgery was performed at least 4 to 6 weeks after completion of the five-week chemoradiotherapy course. Postoperatively, 80 mg/m² of TS-1 was administered every day for 4 weeks with 2 weeks off for 6 months (4 courses).

Table 2 hereinbelow presents the protocol for the preoperative chemoradiotherapy described herein.

TABLE 2
Protocol for the Present Preoperative Chemoradiotherapy

| | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|
| RT 1.8 Gy/day | ↓ | ↓ | ↓ | ↓ | ↓ | | | ↓ | ↓ | ↓ | ↓ | ↓ | | | ↓ | ↓ | ↓ | ↓ |
| CPT-11 90 min iv | ↓ | | | | | | | ↓ | | | | | | | | | | |
| TS-1 bid po | ↓↓ | ↓↓ | ↓↓ | ↓↓ | ↓↓ | | | ↓↓ | ↓↓ | ↓↓ | ↓↓ | ↓↓ | | | | | | |

| | 19 | 20 | 21 | 22 | 23 | 24 | 25 | 26 | 27 | 28 | 29 | 30 | 31 | 32 | 33 | 34 | 35 |
|---|---|---|---|---|---|---|---|---|---|---|---|---|---|---|---|---|---|
| RT 1.8 Gy/day | ↓ | | | ↓ | ↓ | ↓ | ↓ | ↓ | | | ↓ | ↓ | ↓ | ↓ | ↓ | | |
| CPT-11 90 min iv | | | | ↓ | | | | | | | ↓ | | | | | | |
| TS-1 bid po | | | | ↓↓ | ↓↓ | ↓↓ | ↓↓ | ↓↓ | | | ↓↓ | ↓↓ | ↓↓ | ↓↓ | ↓↓ | | |

Dose Titration Schedule

Dose levels were established in the manner shown hereinbelow. TS-1 (80 mg/m²/day) was administered orally for 5 days with 2 days off on Days 1 to 5, 8 to 12, 22 to 26 and 29 to 33. CPT-11 was administered intravenously on Days 1, 8, 22 and 29.

Level 1 (CPT-11, 40 mg/m²) was the starting dose, and dose was titrated until the maximum tolerated dose could be estimated. The CPT-11 dose was increased in 20 mg/m² (two-level) increments until 60 mg/m² and then in 10 mg/m² (one-level) increments thereafter.

After the level of the maximum tolerated dose was confirmed, the recommended dose was determined by reducing the 10 mg/m² (one level) increments.

Maximum tolerated dose was determined on the basis of the adverse reactions observed during the first week after completing chemotherapy. (Maximum tolerated dose was evaluated in the modality of coadministration with radiation.)

TABLE 3

| | TS-1 bid po | | Radiation 1.8Gy/ |
|---|---|---|---|
| | Days 1-5, 8-12, | CPT-11 90 min iv | day × 5 days/ |
| Level | 22-26, 29-33 | Days 1, 8, 22, 29 | wk × 5 wk |
| Level 0 | 80 mg/m²/day | | |
| | 5 days treatment with | 30 mg/m² | 45 Gy |
| | 2 days off per wk | | |
| Level 1 | 80 mg/m²/day | | |
| | 5 days treatment with | 40 mg/m² | 45 Gy |
| | 2 days off per wk | | |
| Level 2 | 80 mg/m²/day | | |
| | 5 days treatment with | 50 mg/m² | 45 Gy |
| | 2 days off per wk | | |
| Level 3 | 80 mg/m²/day | | |
| | 5 days treatment with | 60 mg/m² | 45 Gy |
| | 2 days off per wk | | |
| Level 4 | 80 mg/m²/day | | |
| | 5 days treatment with | 70 mg/m² | 45 Gy |
| | 2 days off per wk | | |
| Level 5 | 80 mg/m²/day | | |
| | 5 days treatment with | 80 mg/m² | 45 Gy |
| | 2 days off per wk | | |
| Level 6 | 80 mg/m²/day | | |
| | 5 days treatment with | 90 mg/m² | 45 Gy |
| | 2 days off per wk | | |
| Level 7 | 80 mg/m²/day | | |
| | 5 days treatment with | 100 mg/m² | 45 Gy |
| | 2 days off per wk | | |

(4) Results

1) Response Rate

The antitumor effect of the preoperative chemoradiotherapy was evaluated by scoring response at the end of the course of therapy according to the Japanese Classification of Colorectal Carcinoma (6^{th} Edition, Japanese Society for Cancer of the Colon and Rectum), and the Japanese JCOG edition of the RECIST (Response Evaluation Criteria in Solid Tumors) Guideline.

Complete response: CR

Partial response: PR

No change: NC

Progressive disease: PD

Not evaluated: NE

Histological response was graded at surgery in subjects that could be graded using the following grades (GR) from the 6th Edition of the Japanese Classification of Colorectal Carcinoma, and included as a measure in evaluation on the effect of preoperative chemotherapy.

Gr-0: No response (Almost no damage such as degeneration or necrosis observed in cancer cells as a result of treatment.)

Gr-1a: Extremely slight response (Degeneration, necrosis, lysis, and the like observed in less than about one-third of the cancer cells.)

Gr-1b: Slight response (Degeneration, necrosis, lysis, and the like observed in at least one-third to two-thirds of the cancer cells.)

Gr-2: Substantial response (Marked degeneration, necrosis, lysis, eradication, and the like observed in at least two-thirds of the cancer cells.)

Gr-3: Complete response (The entire cancer shows necrosis, or shows lysis and eradication. Has been replaced by granulation-like tissue or fibrous foci.)

The results are shown in Tables 4 and 5.

TABLE 4

| Total | CR | PR | NC | PD | NE |
|---|---|---|---|---|---|
| (patients) | (Patients) | (Patients) | (Patients) | (Patients) | (Patients) |
| 18 | 7 | 11 | 0 | 0 | 0 |

TABLE 5

| Total | Gr-3 | Gr-2 | Gr-1b | Gr-1a | Gr-0 |
|---|---|---|---|---|---|
| (Patients) | (Patients) | (Patients) | (Patients) | (Patients) | (Patients) |
| 16 | 8 | 8 | 0 | 0 | 0 |

In Table 4, seven subjects (38.9%) showed complete response (pCR), i.e., eradication of cancer cells upon microscopic examination. Furthermore, an extremely high response rate of (7+11)/18=100% was obtained with grades of PR or better in 18 of 18 subjects. This response rate is superior to that seen with conventional chemoradiotherapy in rectal cancer (e.g., 5-FU/CPT-11/RT).

In Table 5, 8 subjects showed a complete response (Gr-3) histologically at surgery. Moreover, 16 subjects showed a substantial response (Gr-2) or higher-grade response. In the two remaining subjects, the tumors were eradicated with preoperative chemoradiotherapy alone and surgery was not required.

2) Adverse Reactions

Side effects were evaluated according to the National Cancer Institute Common Toxicity Criteria (Version 2.0; NCI-CTC). Effects not on the NCI-CTC form were scored on the following five-grade scale.

| | | |
|---|---|---|
| 0: None | 1: Mild | 2: Moderate |
| 3: Severe | 4: Serious | |

Gr-4 adverse events (hematologic and nonhematologic toxicity) were not seen at up to 80 mg/m² of CPT-11. Almost no Gr-3 adverse events were seen, with only two of 15 subjects showing them (one case of hematologic toxicity and one case of nonhematologic toxicity). All subjects began treatment as in patients, but nine (50 percent) were able to continue treatment as outpatients after the second administration of CPT-11. There was one case of hemotoxicity and one case of nonhemotoxicity of Grade 3 or higher among three subjects at CPT-11 90 mg/m². Accordingly, the final recommended dose of CPT-11 with the present method of therapy was 80 mg/m². Thus, it can be said the present therapy is safe and capable of maintaining the patients' QOL.

1. Chemoradiotherapy for rectal cancer, comprising radiation irradiation in combination with administration of tegafur in a therapeutically effective amount, gimeracil in an amount effective for potentiating the antitumor effect, oteracil potassium in an amount effective for inhibiting side effects, and camptothecin or a camptothecin derivative in a therapeutically effective amount.

2. The chemoradiotherapy according to claim 1, wherein the camptothecin derivative is irinotecan hydrochloride.

3. The chemoradiotherapy according to claim 1, wherein the molar ratio of tegafur, gimeracil, and oteracil potassium is 1:0.1˜5:0.1˜5.

4. The chemoradiotherapy according to claim 3, wherein the molar ratio of tegafur, gimeracil, and oteracil potassium is 1:0.4:1.

5. The chemoradiotherapy according to claim 4, wherein a combination drug of tegafur, gimeracil and oteracil potassium with a molar ratio of 1:0.4:1 is administered in an amount of about 10 to about 200 mg/m²/day, based on the tegafur; irinotecan hydrochloride is administered in an amount of about 10 to about 200 mg/²/day; and the radiation dose is in a range of 40 to 60 Gy per course.

6. The chemoradiotherapy according to claim 5, wherein the combination drug containing tegafur, gimeracil and oteracil potassium at a molar ratio of 1:0.4:1 is administered in an amount of about 80 mg/m²/day, based on the tegafur, on Days 1 to 5, 8 to 12, 22 to 26, 29 to 33; the irinotecan hydrochloride is administered in an amount of about 80 mg/m²/day on Days 1, 8, 22 and 29; and 1.8 Gy/day of radiation is administered on Days 1 to 5, 8 to 12, 15 to 19, 22 to 26 and 29 to 33.

7. The chemoradiotherapy according to claim 1, wherein the disease stage of the patient to undergo treatment is T3 or T4 in terms of depth of wall invasion and falls within a range of N0 to N3 in terms of lymph node metastasis according to the rectal cancer staging system.

8. The chemoradiotherapy according to claim 7, which is performed prior to surgical resection of the tumor.

9. An antitumor agent for use in chemoradiotherapy of rectal cancer, comprising a combination of tegafur in a therapeutically effective amount, gimeracil in an amount effective for potentiating an antitumor effect, oteracil potassium in an amount effective for inhibiting side effects, and camptothecin or a camptothecin derivative in a therapeutically effective amount.

10. The antitumor agent according to claim 9, wherein the camptothecin derivative is irinotecan hydrochloride.

11. The antitumor agent according to claim 9, wherein the molar ratio of tegafur, gimeracil and oteracil potassium is 1:0.1˜5:0.1˜5.

12. The antitumor agent according to claim 11, wherein the molar ratio of tegafur, gimeracil and oteracil potassium is 1:0.4:1.

13. The antitumor agent according to claim 9, which is used for preoperative chemoradiotherapy of rectal cancer.

14. An antitumor agent for use in chemoradiotherapy of rectal cancer, which is a pharmaceutical formulation consisting of two or more preparations containing tegafur, gimeracil, oteracil potassium, and camptothecin or a camptothecin derivative, which preparation each comprises one or any combination of said active ingredients, or which is a pharmaceutical formulation consisting of a single preparation containing all of the active ingredients.

15. The antitumor agent according to claim 14, wherein the camptothecin derivative is irinotecan hydrochloride.

16. The antitumor agent according to claim 14, wherein the molar ratio of tegafur gimeracil and oteracil potassium is 1:0.1˜5:0.1˜5.

17. The antitumor agent according to claim 16, wherein the molar ratio of tegafur, gimeracil and oteracil potassium is 1:0.4:1.

18. The antitumor agent according to claim 14, which comprises a combination drug containing tegafur, gimeracil and oteracil potassium as the active ingredients and a preparation containing irinotecan hydrochloride as the active ingredient.

19. The antitumor agent according to claim 14, which is used for preoperative chemoradiotherapy for rectal cancer.

20. A kit for use in chemoradiotherapy of rectal cancer, which comprises a combination of (a) a combination drug containing tegafur in a therapeutically effective amount, gimeracil in an amount effective for potentiating an antitumor effect, oteracil potassium in an amount effective for inhibiting side effects, and (b) a preparation of camptothecin or a camptothecin derivative in a therapeutically effective amount.

21. The kit according to claim 20, wherein the camptothecin derivative is irinotecan hydrochloride.

22. The kit according to claim 20, wherein the molar ratio of tegafur, gimeracil, and oteracil, potassium is 1:0.1˜5:0.1˜5.

23. The kit according to claim 22, wherein the molar ratio of tegafur, gimeracil and oteracil potassium is 1:0.4:1.

24. The kit according to claim 20, which is used for preoperative chemoradiotherapy for rectal cancer.
